(19) United States
(12) Patent Application Publication
(10) Pub. No.: US 2025/0120924 A1
(43) Pub. Date: Apr. 17, 2025

(54) COMPOSITIONS OF TAURINE AND MALIC ACID AND METHODS OF MAKING THE COMPOSITIONS

(71) Applicant: NutriScience Innovations LLC, Milford, CT (US)

(72) Inventor: Michael Lelah, Arlington Heights, IL (US)

(21) Appl. No.: 18/432,775

(22) Filed: Feb. 5, 2024

Related U.S. Application Data
(63) Continuation-in-part of application No. 18/533,368, filed on Dec. 8, 2023.
(60) Provisional application No. 63/544,001, filed on Oct. 13, 2023.

(51) Int. Cl. A61K 31/145; A61K 9/16; A61K 31/191; A61P 25/26

(52) U.S. Cl. CPC A61K 31/145; A61K 9/1611; A61K 9/1652; A61K 31/191; A61P 25/26

(57) ABSTRACT

The present disclosure provides a novel supplement composition comprising a 2:1 molar ratio of taurine to malic acid, which can be added to food or beverages, or incorporated as a dietary supplement. The present supplement composition when added to a food, beverage or dietary supplement provides various health benefits, including ergogenic effects of enhanced physical performance, stamina, and recovery, threshold pain reduction, increased power, and increased vasodilation for improved recovery and performance. The present disclosure also provides method of making the composition.

### TECHNICAL FIELD

The present disclosure generally relates to a unique composition containing a combination of taurine and malic acid, and methods and processes of making the composition. The present disclosure specifically relates to an improved dietary supplement composition, including a supplement composition that can be added to human and animal food products, beverages, pharmaceutical and nutraceutical formulations to create dietary supplements, all having a unique composition of taurine and malic acid to provide various health benefits, including ergogenic effects of enhanced physical performance, stamina, and recovery. The present supplement compositions are beneficial for supporting an active lifestyle and nutrition.

### BACKGROUND

Taurine, or 2-aminoethanesulfonic acid is an amino acid which is essential for cardiovascular function, skeletal muscle development and function, and for the functioning of the central nervous system. It is an osmolyte which regulates cell volume, functioning as an antioxidant, and providing various health benefits. New research suggests that taurine may slow the aging process. Taurine improves physical performance, including for example, delaying the onset of physical fatigue during exercise.

Taurine increases mitochondrial biogenesis and thus plays a role in aerobic capacity during exercise. It has known uses in energy beverages and in dietary supplements to increase energy and assist in muscle recovery when used up to about 1,000 mg/day human dose. Higher doses (up to 6-10 g/day) of taurine alone (not in combination with other ingredients), have been studied for its aerobic and anaerobic performance and metabolic outcomes.

Malic acid is an organic acid. It is available in the D,L-malic acid form, as a 50/50 racemic mixture of D-malic acid, and also as L-malic acid. Since malic acid is achiral, the two forms are indistinguishable, and thus the term malic acid as used herein should be understood to cover both forms or combinations thereof. Malic acid is used as a pH adjuster, flavor enhancer or preservative in foods and beverages. It is used in beverages, including in energy drinks, for these purposes.

L-malic acid is the naturally occurring form of malic acid. In this form it is a Krebs cycle intermediate, it increases availability of NAD+, which is critical for generation of ATP (energy). L-malic acid also increases NADPH, which is a fundamental antioxidant in the mitochondrial metabolic pathway, and it helps with exercise recovery as it counters the build-up of lactic acid.

Combinations of taurine and malic acid for energy enhancement and for recovery health benefits have been described. The taurine and malic acid compositions are mixtures, wherein the malic acid is only used as a pH modifier, and/or the amount of taurine has been less than 1,000 mg dose per day. Additionally, malic acid is not included for ergogenic effects or active nutrition. Additionally, the amounts of taurine used in energy drinks are generally below those levels required for a health effect.

Therefore, a need exists for a health and energy beverage, food or dietary supplement composition whereby the amount of taurine in the composition is greater than 1,500 mg/day, up to about 3,500 mg/day. A need also exists for a unique synergistic combination of taurine and malic acid in order to provide health benefits, including ergogenic health benefits, such as an increase in exercise performance and power, improved energy and recovery, muscle damage prevention, and inflammation reduction, among other benefits. In a desired composition, taurine and malic acid synergistically combine for the intended effect and desired benefits.

### SUMMARY

A composition of taurine and malic acid in the range of 2.1:1 molar ratio to 1.9:1 molar ratio (calculated to the range of 1.91:1 to 1.82:1 mass ratio, or the range of 33.79% to 36.06% by weight malic acid to 2 decimal places), is provided. A supplement composition of taurine and L-malic acid of 2:1 molar ratio (calculated to 1.87:1 mass ratio, or 34.89% L-malic acid by weight to 2 decimal places), is provided.

A supplement composition where a salt is made by titration of the combination of taurine and malic acid with an alkali to neutrality is provided.

A method of preparing a solid composition wherein taurine and malic acid are combined, dissolved in water and then dried using a conventional drying process, is provided.

A method of preparing a solid composition comprising the steps of mixing taurine and malic acid powders in the 2:1 ratio, and then performing wet agglomeration (e.g. Glatt® agglomeration) on the mixture using various methodologies such as spray agglomeration, spray granulation, high shear granulation or encapsulation, to create the solid composition, is provided.

A method for preparing a solid composition consisting of preparing a slurry consisting of between 10% and 90% solids with agitation, and then drying this slurry using a conventional drying process, is provided.

A daily human dose of a composition of about 1,500 mg to 3,500 mg taurine and about 700 mg to 1,700 mg malic acid, for use as a health and active lifestyle nutrition ergogenic, energy and muscle recovery supplement composition delivered in a beverage, food or as a dietary supplement for human use is provided.

Based on a standard human weight of approximately 70 kg, a daily dose of a composition comprising about 20 to 50 mg/kg body weight taurine and about 10 mg/kg to 25 mg/kg body weight malic acid, for use as a health and active lifestyle nutrition energy and recovery supplement composition delivered in a beverage, food or as a dietary supplement for a mammal, is provided. The human to mammal conversion is based on body weight and can be adjusted for other factors including metabolism and surface area.

A dosage form for administrating the disclosed dietary supplement composition of taurine and malic acid, the dosage form includes a beverage, capsule, softgels, tablet, lozenge, gummy, stick pack, powder pack, etc, is provided.

A supplement composition for use in a food, beverage or as a dietary supplement, the composition comprising taurine and malic acid combined with other ergogenic and active nutritional ingredients such as caffeine, creatine, whey protein, betaine, nitrates, proteins, etc., is provided.

A supplement composition for use in a food, beverage or as a dietary supplement, the composition comprising taurine and malic acid combined with other excipients such as maltodextrin, gum Arabic, silicon dioxide etc., is provided.

### BRIEF DESCRIPTION OF THE DRAWINGS

FIG. 1 illustrates loss on drying (LOD) analysis of the compositions according to the present disclosure;

FIG. 2 illustrates a melting point analysis for the compositions according to the present disclosure;

FIG. 3 illustrates a titration assay and analysis of the amount of sodium hydroxide solution consumed by the compositions according to the present disclosure;

FIG. 4 illustrates a nematode animal model used to evaluate lifespan/survival and energy/activity when dosed with the compositions according to the present disclosure; and,

FIG. 5 illustrates energy/activity levels of the neamtode animal model measured at an optimum 15 days of life when dosed with the compositions according to the present disclosure.

### DETAILED DESCRIPTION

The present disclosure provides an improved and unique dietary supplement composition providing various health benefits, including ergogenic effects of enhanced physical performance, including increased strength, stamina and recovery, threshold pain reduction, and increased vasodilation for improved recovery and performance. Specifically, the present disclosure relates to a unique dietary supplement composition comprising an approximate 2:1 molar ratio of taurine to malic acid. This specific combination has not been previously described. The present composition can be incorporated into a food, beverage or as a dietary supplement.

Malic acid is a dicarboxylic acid. The disclosed specific ratio of the present composition is approximately 2:1 taurine to malic acid, when mixed in an aqueous solution and then dried, creating a unique molecular structure in the solid phase wherein one molecule of taurine associates with each carboxylic group in one molecule of malic acid, accounting for a novel combination of the two ingredients. This specific ratio range imparts unexpected unique physical and chemical properties to the composition in the solid state, which have not been previously described. The present composition also exhibits unique properties when the solid composition thus formed is subsequently dissolved in water. Specifically, the approximate 2:1 molar composition in the solid phase, when consumed in the body, synergistically improves energy and recovery, among other health benefits.

In the Examples below, it is calculated that a 2:1 molar ratio of taurine to malic acid is 1.87:1 mass ratio (to two decimal places) of taurine to malic acid based on the known molecular weights of 125.14 for taurine and 134.09 for malic acid. This results in approximately 34.89% by weight malic acid (to 2 decimal places) in the formula.

The known method of preparation of a 2:1 molar ratio of taurine to malic acid is to physically mix solid taurine and solid malic acid in a 2:1 molar ratio. However, this known method of combination does not impart any unique characteristics or properties. The method of preparing the composition of present disclosure is novel in that when taurine and malic acid are dissolved in water after solid mixing at the approximately 2:1 molar ratio, and then evaporated to dryness, the resulting solid composition unexpectedly displays unique physical and chemical properties. Notably, as long as the molar ratio was between and within 1.9:1 and 2.1:1, a small degree of variation, the unexpected health properties were observed when the present composition was administered to a subject.

Although the disclosed method of preparing the novel composition of the present disclosure, the method requires a considerable amount of water and therefore a considerable amount of energy to dry. Therefore, a need exists to develop a method for preparing the unique solid combination of taurine and malic acid starting with minimal amount of water which will require less energy to dry. A slurry with between 10% and 90% solids is effective for subsequent drying. Some agitation of the slurry was necessary to keep the solids suspended in water during processing. The slurry prepared within the molar range of 2.1 to 1.9:1 as previously described, also exhibited the same unexpected physical and chemical properties.

Various methods of drying, including freeze drying, spray drying, fluidized bed drying, drum drying, infrared drying, vacuum drying, etc. may be utilized for the final drying step. Various excipients, such as maltodextrin, gum Arabic, silicon dioxide may be added to facilitate drying.

Separately, a liquid composition prepared by dissolving taurine and malic acid in water without first drying to the solid state, and then dissolving in water does not impart any unique properties otherwise provided by the present composition prepared by the preferred method.

After preparing a composition of taurine and malic acid, and then drying, as described above, and then subsequently dissolving the solid composition in water, this soluble composition unexpectedly also exhibited unique chemical properties between the narrow range of 1.9 to 2.1 taurine to 1 malic acid molar ratio. It was not expected that the solid composition when dissolved in water would continue to exhibit unique properties as described below in the examples.

In summary, the combination of taurine and malic acid, in a narrow 1.9:1 to 2.1:1 molar ratio range, in aqueous solution, followed by drying, created a unique composition with unique properties. This could be obtained from a solution of taurine and malic acid, or from a slurry up to 90% solids. Using other molar ratios of taurine to malic acid, or without first drying to a solid, did not impact these unique properties or characteristics. The unique molecular interactions between taurine and malic acid are the result of the disclosed specific process conditions.

In addition to the compositions and method of making the compositions combining taurine and malic acid as described above, the present disclosure further includes a combination of taurine and malic acid in the described 2:1 molar ratio, in the form of a salt. This salt is made by titrating to neutral pH a mixture of taurine and malic acid in the approximately 2:1 ratio, with an alkali, such as sodium or potassium hydroxide. This creates a salt form (disodium or dipotassium salt, for example) which can be dried and rehydrated using the methods described above.

As noted previously, the commercial product dose for taurine in humans is typically less than 1,000 mg/day in an energy formula. For anti-aging, human doses of taurine have been calculated to be greater than 5 grams/day. However, the known safe upper limit of taurine is about 3,500 mg per day human dose and this limits the safe use of taurine in commercial products to about 3,500 mg/day. The reason for this upper limit is that based on toxicity and safety studies, it has been concluded that a safe range of taurine is up to about 3 grams per day.

Therefore, a need exists for a human health, ergogenic, or energy beverage, food or dietary supplement composition whereby the amount of taurine in the composition is greater than 1,500 mg/day, up to about 3,500 mg/day. The amount of malic acid in this same composition is between about 700 mg/day to 1,700 mg/day. The total dose of the composition would be in the range of 2,200 mg/day to about 5,200 mg/day. An animal dose may be calculated from the human dose according to previous methodologies. A typical animal dose would be in the range of 20 to 50 mg/kg body weight per day. The amount of malic acid in this same composition is approximately 10 mg/kg to 25 mg/kg body weight per day. The total dose of the composition would be in the range of 30 mg/kg to 75 mg/kg body weight per day.

### Examples

EXAMPLE 1—Preparation of solid compositions of taurine and malic acid: Solid compositions of taurine and L-malic acid were prepared according to Table I below. Weighed amounts of taurine and malic acid were dissolved in water, mixed and then freeze dried.

TABLE I
Compositions

| Composi- | Molar Amount | Molar Amount | % L-malic acid by weight |
|---|---|---|---|
| tion # | of Taurine | of L-malic acid | (to 2 decimal places) |
| 1 | 1.5 | 1.0 | 41.67% |
| 2 | 1.9 | 1.0 | 36.06% |
| 3 | 1.95 | 1.0 | 35.46% |
| 4 | 2.0 | 1.0 | 34.89% |
| 5 | 2.05 | 1.0 | 34.33% |
| 6 | 2.1 | 1.0 | 33.79% |
| 7 | 2.5 | 1.0 | 30.00% |

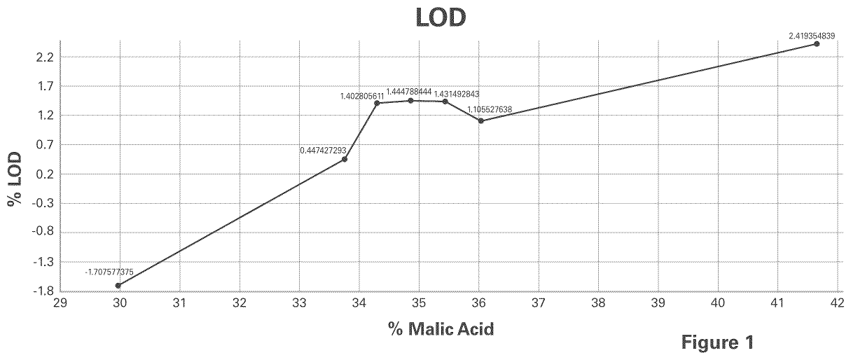

EXAMPLE 2—Loss on Drying: Loss on drying (LOD) was evaluated by preparing the compositions according to Example 1 and then performing an LOD test in an open crucible at 106° C. for 3 hours using standard methodology. The results are shown in FIG. 1.

The results show an unexpected pertubation in LOD within the range of 33.79% to 36.06% malic acid for the solid compositions #2 to #6. This indicates unexpected unique molecular associations in this molar range.

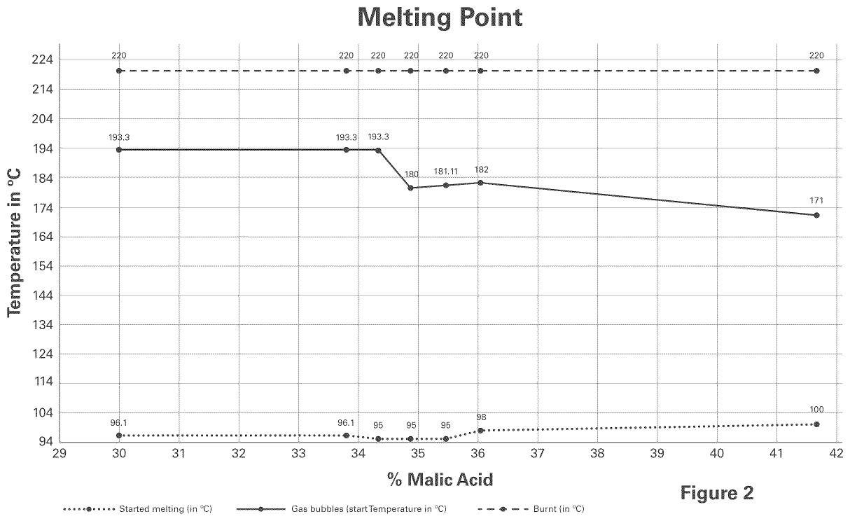

EXAMPLE 3—Melting Point. The melting point for the compositions in Example 1 were obtained using a standard manual melting point apparatus. The melting points and observations are listed in Table II below and FIG. 2.

TABLE II
Melting points and observations for the
compositions in Example 1 Melting point

| S. No. | Concentration % | Comments |
|---|---|---|
| 1 | 1.5:1 | 1. Started melting at 100° C. |
| | | with Gas bubbles from 171° C. |
| | | to 218.3° C. |
| | | 2. Not completely melted but |
| | | burnt at 220° C. |
| 2 | 1.9:1 | 1. Started melting at 98° C. |
| | | with gas bubbles till 182° C. |
| | | 3. Burnt at 220° C. |
| 3 | 1.95:1 | 1. Started melting at 95° C. |
| | | with gas bubbles from |
| | | 181.11° C. |
| | | 3. Burnt at 220° C. |
| 4 | 2:01 | 1. Started melting at 95° C. |
| | | with gas bubbles till 180° C. |
| | | 3. Burnt at 220° C. |
| 5 | 2.05:1 | 1. Started melting at 96° C. |
| | | 2. Melting with gas bubbles |
| | | at 193.3° C. |
| | | 3.Burnt at 220° C. |
| 6 | 2.1:1 | 1. Started melting at 96° C. |
| | | with gas bubbles 193.3° C. |
| | | Started to burn at 220° C. |
| 7 | 2.5:1 | Started melting at 96° C. with |
| | | gas bubbles 193° C. |
| | | Started to burn at 220° C. |

The melting point results show unexpected behaviors between 1.9 and 2.1 molar ratios of taurine to malic acid (solid compositions #2 through #6 from Table I). These results again indicate unexpected pertubations in the results which may be explained by unexpected molecular associations in these ranges.

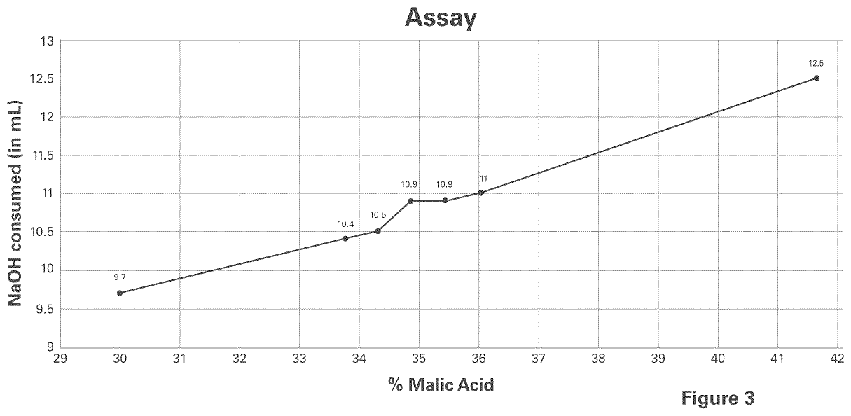

EXAMPLE 4—Titration assay: The solid compositions #1 through #7 from Example 1 were dissolved in water at 1 gram of the solid composition in 20 ml of water. This solution was then titrated against a solution of 1M sodium hydroxide to an end pH of 8.0 using an electronic pH detector (modified USP method for malic acid). The amount of sodium hydroxide solution consumed was recorded. The results are shown in FIG. 3.

The results of this experiment show an unexpected perturbation in the assay between 34.33% malic acid (composition #5) and 35.46% (composition #3), again indicating unexpected results in a narrow molecular range, even after the solid composition was dissolved in water. It also appears that the perturbations in amount of sodium hydroxide required are slightly broader—in the previously identified range 2.1 to 1.9 molar ration taurine to malic acid; however, the assay sensitivity was limited.

EXAMPLE 5—Slurry Compositions: Slurry compositions of 2:1 molar ratio taurine to malic acid were made at 20%, 40%, 60%, 80% solids in water. In order to assess processing capabilities, these slurries were left to sit over time. Visual clear liquid at the top was observed after 30 seconds, 60 seconds, 90 seconds, and 30 minutes respectively. All the slurries maintained their visual homogeneity for at least 30 minutes with continuous agitation. This means that the slurries can be processed in drying equipment. The 80% slurry was freeze dried. After freeze drying, LOD, melting point and titration assay matched that for composition #4, indicating that a slurry could be used to achieve the same form as in the examples above.

EXAMPLE 6—Taurine and L-malic acid powders were mixed in a ratio 2:1 molar ratio and treated by wet agglomeration on a lab scale Glatt® agglomerator using water. The resultant dry powder was a single particle type powder indicating that the taurine and malic acid combined or fused together during the wet agglomeration. The assay results using sodium hydroxide titration are shown in Table III below:

TABLE III

| | Sample Wt. | Water | 1N NaOH | | % L-Malic |
|---|---|---|---|---|---|
| | (mg) | (ml) | consumed (ml) | End pH | acid |
| Test 1 | 2000 | 40 | 10.7 | 8.0 | 35.7 |
| Test 2 | 2000 | 40 | 10.9 | 8.0 | 36.4 |
| Blank | 0000 | 40 | 0.05 | 8.0 | 0 |

The percentage of L-malic acid in the resulting powder formed through wet agglomeration is predicted as 34.9%. The results shown in Table III fall within a +/−5% variability due to standard processing and testing variability.

Water activity was measured for the premix (taurine and L-malic acid physical powder mixture) at 0.26 and for the agglomerated mixture at 0.28. These values are similar and indicate a high level of dryness of the powders before and after wet agglomeration.

The results demonstrate this process method using wet agglomeration produces a 2:1 molar ratio composition of taurine and L-malic acid as a single particle dry powder.

EXAMPLE 7—Other Combinations: A powder mix of taurine powder and malic acid was made according to composition #4. The LOD was determined to be 0.72% and the titration assay was determined to be 10.8 ml of NaOH. These results are different from the results in Examples 2 and 3 indicating that just mixing taurine and malic acid powders together in the solid state, or just dissolving taurine and malic acid directly in water without first going through the drying method, did not produce the unexpected results previously described.

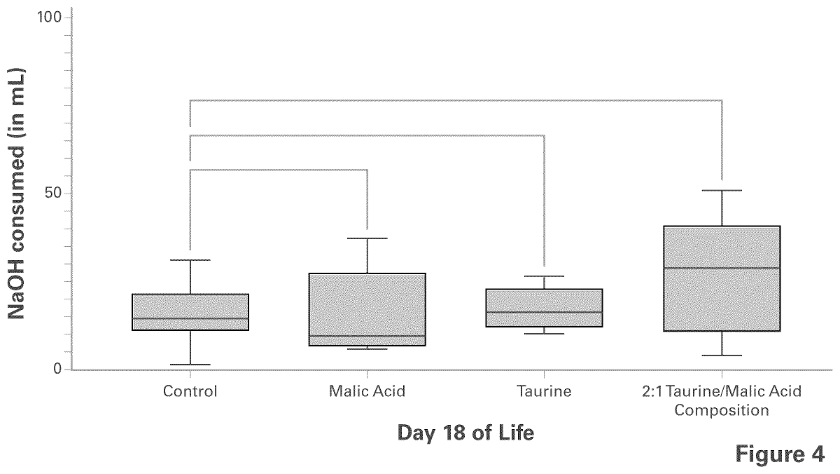

EXAMPLE 8—Animal Studies: A nematode animal model was used to evaluate lifespan/survival and energy/activity. Wild-type nematodes were loaded into microfluidic chips and were fed 20 ng/ml E. coli OP50 in NGM buffer food source. The methods have been previously described. Nematode lifespan was measured by survival at standard 18 days, while nematode activity was measured at an optimum 15 days using a visual score. Dosing of 2:1 taurine to malic acid molar ratio composition (composition #4, Example 1) was made using the methodology described previously was at 200 micrograms/ml/day, with the solid composition being dissolved in the aqueous feed. This is equivalent to a human dose of 5 grams per day for these animals. The 5 gram per day human dose equivalent represents a dose of 3.26 grams of taurine and 1.74 grams of malic acid (2:1 molar ratio). The results were compared with taurine alone (human equivalent dose 3.26 grams), and malic acid alone (1.74 grams malic acid). The nematode survival results are shown in FIG. 4.

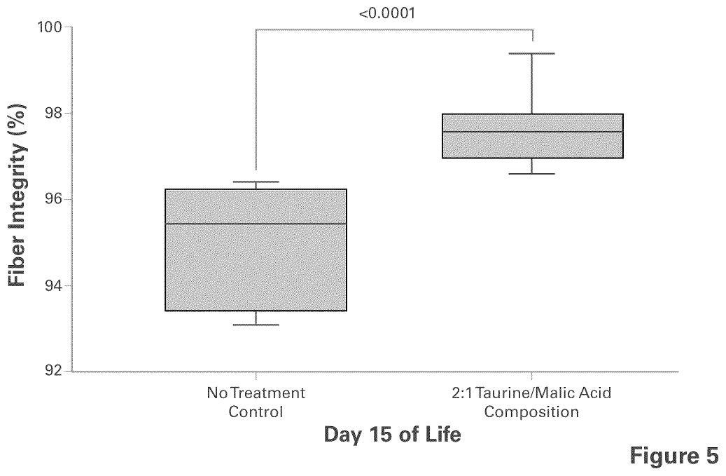

Eighteen days is the typical survival time for nematodes. Eighteen days may be considered “old-age” for these animals. The results at 18 days show that malic acid alone or taurine alone did not improve survival times versus control. However, 2:1 taurine/malic molar ratio (composition #4) synergistically improved survival time versus the malic acid and taurine components and versus the control. This unexpected synergistic effect has not been previously observed. Nematode survival is a measure of age-related health and is related to antioxidant effects and other biochemical pathway modifications. The synergistic effect of combining taurine and malic acid in Composition #4 results in unexpected increased health benefts. Specific energy/activity levels of the neamtodes was measured at an optimum 15 days of life (which may be considered “middle-age”). FIG. 5 shows the results under the same experimental conditions.

In this evaluation, 2:1 molar taurine/malic acid (composition #4) at a human equivalent dose of 5 grams per day showed statistically significant improvement in activity over control. Activity is a measure of worm energy and indicates an improvement in energy on consumption of the composition.

These animal studies show that the 2:1 composition of taurine to malic acid, at an equivalent dose of 5 grams per day (3.26 grams/day taurine plus 1.74 grams per day malic acid), demonstrate improved health benefits with respect to aging and energy.

1. A supplement composition comprising an approximate 2:1 molar ratio of taurine to malic acid.

2. The supplement composition of claim 1, wherein the composition provides a human dose of approximately 5 grams per day.

3. The supplement composition of claim 1, wherein the composition provides an animal dose of approximately 70 mg/kg body weight per day.

4. The supplement composition of claim 2, wherein the composition comprises approximately 3.26 grams/day taurine plus 1.74 grams per day malic acid.

5. The supplement composition of claim 1, wherein the composition provides a human dose of between 2.2 grams and 5.3 grams per day.

6. The supplement composition of claim 1, wherein the composition provides an equivalent animal dose of between 30 mg/kg and 75 mg/kg body weight per day.

7. The supplement composition of claim 1, wherein one molecule of taurine which contains two carboxyl groups is combined with approximately two molecules of malic acid, creating a synergistic combination of the two ingredients.

8. The supplement composition of claim 1, wherein the composition is further combined with active nutritional ingredients selected from caffeine, creatine, whey protein, betaine, nitrates, proteins.

9. The supplement composition of claim 1, wherein the composition is further combined with excipients selected from maltodextrin, gum Arabic, silicone dioxide.

10. The supplement composition of claim 1, wherein the composition is useful as an additive in a beverage and/or a food product.

11. The supplement composition of claim 1, wherein the composition is used as a dietary supplement.

12. The supplement composition of claim 1, wherein the composition provides a plurality of health benefits including improvements to aging and improvements to energy levels when added to a beverage and/or food product or administered as a dietary supplement to a mammal.

13. A method of producing a supplement composition for use as a dietary supplement, or as an additive in a food or beverage product, the method including the steps of:
combining weighed amounts of taurine powder and malic acid powder;
dissolving the combined taurine powder and malic acid powder in water creating a slurry;
mixing the slurry;
drying the slurry;
wherein the resulting dietary supplement, food or beverage composition comprises an approximately 2:1 molar ratio of taurine to malic acid.

14. The method of producing the supplement composition of claim 13, wherein the slurry has a composition of between 10% and 90% solids.

15. The method of producing the supplement composition of claim 13, wherein the method further includes the steps of adding into the slurry an excipient selected from maltodextrin, gum Arabic or silicone dioxide.

16. A method of producing a dietary supplement composition comprising the steps of:
providing a solution of taurine and malic acid in an approximately 2:1 molar ratio;
titrating the solution to a neutral pH with an alkali; and,
creating a salt.

17. The method of producing the dietary supplement composition of claim 16, wherein the alkali is one of sodium or potassium hydroxide.

18. The method of producing the dietary supplement composition of claim 16, the method further includes the steps of adding into the solution an excipient selected from maltodextrin, gum Arabic or silicone dioxide.

19. A method of producing a dietary supplement composition comprising the steps of:
combining weighed amounts of taurine powder and malic acid powder;
treating the combined taurine powder and malic acid powder using wet agglomeration;
creating a single particle dry powder having a 2:1 molar ratio of taurine to malic acid.
